(19) United States
(12) Patent Application Publication
(10) Pub. No.: US 2022/0145234 A1
(43) Pub. Date: May 12, 2022

(54) Closed Tissue Disaggregation and Cryopreservation

(71) Applicant: Asymptote Ltd., Cambridge (GB)

(72) Inventors: George John Morris, Cambridge (GB); Stephen Lamb, Cambridge (GB)

(21) Appl. No.: 17/433,463

(22) PCT Filed: Feb. 28, 2020
(86) PCT No.: PCT/EP2020/000053

§ 371 Date: Aug. 24, 2021

(30) Foreign Application Priority Data
Mar. 1, 2019 (GB) 1902763.0
Mar. 27, 2019 (GB) 1904249.8

(51) Int. Cl. C12M 1/33; C12M 1/00

(52) U.S. Cl. CPC C12M 45/02; C12M 23/14; C12M 23/38; C12M 45/20; C12M 23/26

(57) ABSTRACT

Disclosed is a device (100, 200) for the disaggregation of tissue samples into individual cells or cell clumps in a closed flexible tissue sample bag (10); the device including two or more resilient feet (134/136, 234/236) which tread sequentially a tissue sample bag receiving area (148,248). Also disclosed is a heat transfer plate (150, 250) for transferring heat energy to or from the area (148,248), the plate having one plate surface (151,251) adjacent the area (148,248) and an opposing surface (152,252) exposed to external thermal influence which faces away from the area (148,248). Further disclosed is a tissue sample receiving bag (10) comprising one or more flexible plastics cavity (12) formed from two layers of the plastics sealed around their edges to form a generally rectilinear periphery with the cavity or cavities (12) within the periphery, and at one side of the periphery is formed one or more sealable access ports (16). One part of the bag is left unsealed to provide a tissue sample receiving opening.

### TECHNICAL FIELD

The present invention relates to apparatus and methods for disaggregation of tissue in a closed volume and to apparatus and methods for thermal control of disaggregated tissue.

### BACKGROUND

In many areas of medicine and biology there is a need to take tissue samples and disaggregate them into cell clumps and single cells for further processing. The number of applications is large and includes extraction of cells, for example:
a) “Primary cells” may be extracted from tissue such as liver, which can be then used in various assays commonly called high throughput screening;
b) Tissue Infiltrating Lymphocytes (TIL) may be extracted from tumour tissue and used as the basis for an autologous cell therapy;
c) Cord tissue may be used to extract mesenchymal stromal cells;
d) Tumours may be excised and their cells analysed for “neoantigen”; and
e) Tissue may be dislocated and cells can be examined, whereby the so-called multi-omics of cells (e.g. proteomics, genomics, epigenomics) may be investigated for many purposes including personalised medicines.

In many applications it is desirable to maintain as many healthy cells as possible, and to keep them in a clean, sterile condition. In this application closed, aseptic, sterile and like terms are intended to mean the condition whereby biological material is separated from its surroundings, but not necessarily wholly free of a bioburden or other contamination, merely free enough that such bioburden or other contamination, if any, does not have a significant influence on the viability or usability of the material which is disaggregated.

One technique of tissue disaggregation of cells is known from WO2018/130845, the contents of which are incorporated herein by reference, as if the wording was repeated herein. In that application, an aseptic tissue processing method, kit and device is disclosed for disaggregation of solid tissue to derive eukaryotic cells into either single cells or small cell number aggregates. The disclosure also describes a semi-automatic aseptic tissue processing method. It is explained in WO2018/130845 that the conditions during solid tissue disaggregation and time taken to harvest the cells have a substantial impact on the viability and recovery of the final cellularised material. A kit is proposed, which together with hardware can introduce enzymes into a hanging bag to aid disaggregation, the kit including a separate bag into which can be pumped a disaggregated sample and a cryoprotectant for freezing after initial cooling.

U.S. Pat. No. 6,439,759 describes a kneading device which includes an internal baffle to aid mixing a closed bag of materials, but the thermal control of this arrangement is not considered.

With that background the inventors of the present invention have realised that there is a need to disaggregate cells taking into account more parameters than have been considered in WO2018/130845, to improve the performance of the disaggregation, freezing and thawing processes, particularly thermal control during such processes, which are not addressed in WO2018/130845 or U.S. Pat. No. 6,439,759.

### SUMMARY OF INVENTION

The present invention concerns apparatus in the form of a treading device for effective disaggregation of tissue into individual cells or cell clumps, typically mammalian cells, and addressing the need for improved thermal control during the disaggregation process. The present invention according to another aspect concerns a thermal control method used with the above-mentioned treading device(s) as well as subsequent disaggregated tissue processing steps. The present invention according to another aspect concerns a disposable flexible container, for example a bag, adapted for use in the devices mentioned above. The above-mentioned aspects are represented in the claims appended herein. More advantages and benefits of the present invention will become readily apparent to the person skilled in the art in view of the detailed description below which provides examples of the invention.

### DRAWINGS

The invention will now be described in more detail with reference to the appended drawings, wherein:

FIG. 1 shows a front view of a treading device for the disaggregation of tissue into individual cells or cell clumps within a closed sample container;

FIGS. 2 and 3 show the device of FIG. 1 in two different respective operational positions;

FIG. 4 shows a plan view of the device shown in the previous Figures;

FIG. 5 shows another plan view of an alternative construction of the device;

FIGS. 6, 7 and 8 show three different constructions of a sample container suitable for use with the device of FIGS. 1 to 5,

FIG. 9 shows a sample bag being prepared for use;

FIGS. 10, 11a, 11b, and 11c show alternative ways of sealing the sample bag;

FIGS. 12, 13 and 14 show apparatus and techniques for preparing the bag for use;

FIG. 15 shows loading of the sample bag or container into the treading device;

FIGS. 16, 17 and 18 show apparatus for dividing a disaggregated sample;

FIGS. 19, 20 and 21 show apparatus for controlling the temperature of a sample or divided sample; and

FIGS. 23 to 25 show a further embodiment of a treading device.

### DETAILED DESCRIPTION

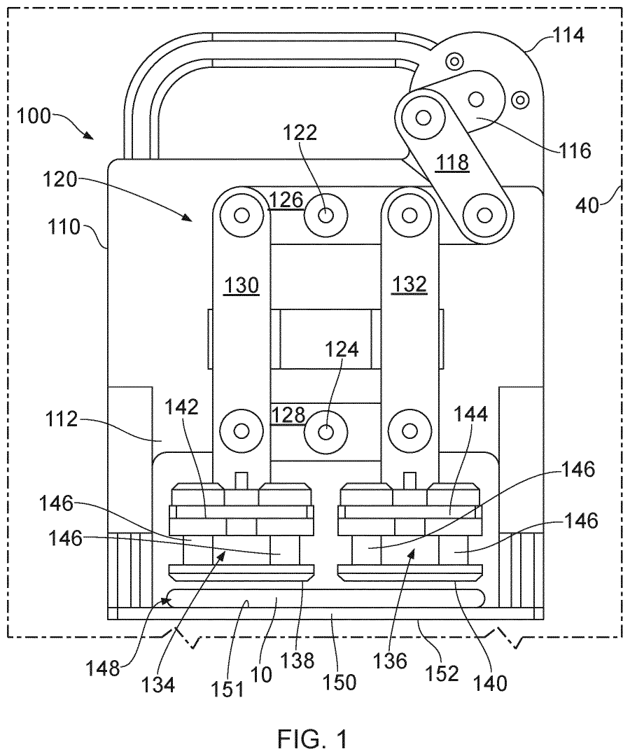

Referring to FIG. 1 there is shown a treading device 100 for the disaggregation of tissue into individual cells or cell clumps within a closed and at least initially aseptic generally flat-sided and relatively thin sample container bag 10. The device includes a housing 110 formed from an assembly of parts that can be removably inserted into a temperature controlled device such as a controlled temperature rate change freezer, thawer or warmer, for example a commercially available freezer known as Via Freeze™, or any other device which provides a controlled rate change in temperature, shown schematically in FIG. 1 and described herein generally as freezer 40. In practice the housing will include a cover, which is not illustrated. In use the device and bag provide a closed system, to disaggregate tissue e.g. excised tumours, parts of excised tumours or needle biopsies etc, and to then cryopreserve the resulting cell suspension for subsequent analysis without the need to transfer the disaggregated sample out of the bag 10.

The housing 110 has a chassis 112 to which is attached a motor unit 114 which includes an electric motor and gearbox, which has an output speed of 10-300 rpm. The output shaft of the motor and gearbox 114 has a crank 116 which drives a connecting rod 118, which in turn is pivotably connected to a treading mechanism 120, which will be moved through one treading cycle for each revolution of crank 116, i.e. a treading cycle between 0.2 and 6 seconds. In more detail this treading mechanism has a parallelogram four bar linkage, which includes two spaced pivots 122 and 124 rigidly mounted to the chassis 112 which pivotably mount two opposed parallel horizontal bars 126 and 128 respectively. Each of the horizontal bars has two parallel treading bars 130 and 132, pivotably connected thereto one on each side of the pivots 122 and 124, together forming the parallelogram linkage. The connecting rod 118 is conveniently pivotably held to an extension of the top horizontal bar, such that moving of that extension causes cyclic up and down motion (in the orientation shown) of the treading bars 130 and 132. To each treading bar 130 and 132 is connected a foot assembly 134 and 136 which, by virtue of the above-mentioned cyclic motion, will move up and down with motion of the crank 116, in a sequentially manner, i.e. when one foot is up the other will be down and vice versa.

The foot assemblies 134 and 136 each include a flat faced sole plate 138 and 140 each plate being spring-mounted to a upper foot frame 142 and 144 respectively, by coiled metal springs 146. In the arrangement described above, or an equivalent arrangement if used, the springs 146 are preloaded. In this case the combined preload is preferably 40-80N, more preferably 30-70 N for each foot preferably about 60N. The combined spring rate is 1-5 N per mm of travel, preferably about 3N per mm, and the intended foot travel is about 8-12 mm, preferably about 10 mm. In addition the surface area of each foot is intended to be about 20 to 50 cm², preferably about 35 cm². This results in a notional, pressure on the bag of between zero (when the foot lifts off the bag or has substantially no load, and up to about 6 N/cm2 (about 9 psi): The preferred notional pressure is about 2N/cm² (about 3 psi). However, given that the bag may not, at least at the start of the treading process, contain a homogeneous material, then there will be lumps of material where the force exerted will be concentrated , and so the pressure is described as ‘notional’ which is the idealised situation, for example to provide a minimum pressure resistance of the bag 10 exerted toward the end of the treading process.

At the bottom of the chassis is a receiving area 148 for the flexible bag 10 and adjacent the receiving area 148 is heat transfer plate 150. The area 148 is large enough to admit the sample processing bag 10 slidable onto the plate 150 via the front of the chassis (the front being shown in FIG. 1). The plate includes an upper surface 151 on which the bag 10 sits, and a lower surface 152 which in use is exposed for externally influenced heating or cooling. The upper surface 151 is generally parallel to the sole plates 138 and 140 of each foot, so that the sole plates move generally parallel to the surface 151. Put another way, the flat sole plates move in a generally perpendicular direction to the surface 151, which prevents significant side forces on the mechanism 120. The plate 150 is formed from metal, preferably aluminium or copper or gold or silver, or alloys containing those metals. Heat conductance is preferably above 100 and more preferably above 200 W/m K measured at 20 degrees Celsius. The thickness of the plate 150 material is about 3 mm or less and provides low thermal mass and thus a quicker reaction of the contents of the bag 10 to follow temperature changes on the opposite side of the plate.

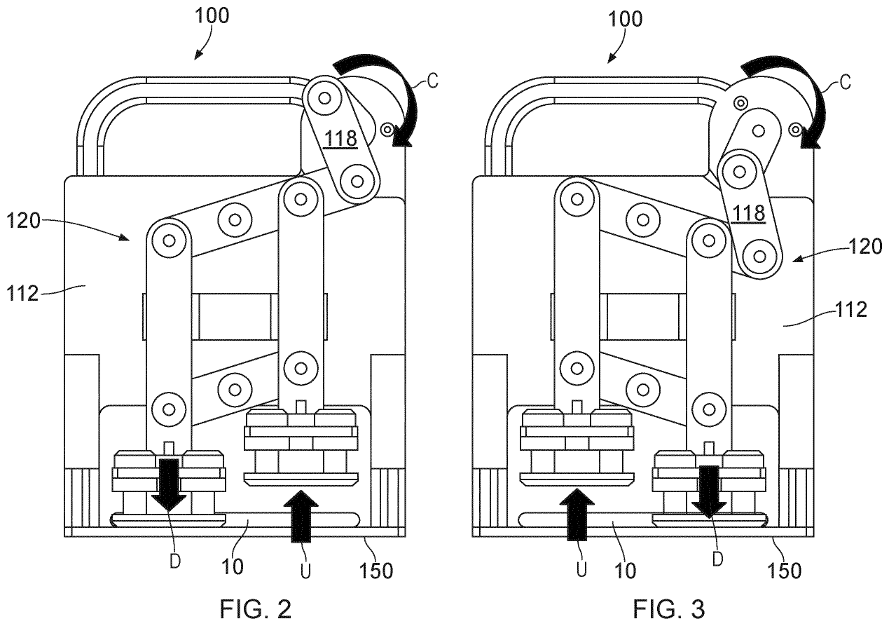

With reference additionally to FIGS. 2 and 3, the device is operated by supplying electrical current to the motor unit 114, to drive the crank 116, in this example clockwise as shown by arrows C. The crank causes the connecting rod 118 to operate the above described treading mechanism 120. It will be noted that the top and bottom of the stroke of the crank, where maximum force is applied to the mechanism 120 coincides with the lowermost position of each foot assembly 134 and 136. The foot assemblies move up and down in the direction of arrows U and D to massage the sample bag 10 sequentially, such that the contents of the bag 10 have an opportunity to move to one side away from the respective treading foot. Since the potentially solid tissue samples in the bag can move away from the treading foot, and because the sole plates 138 and 140 of each foot are sprung loaded, with additional resilient travel being afforded to the feet even when they are at the bottom of their stroke, then there is less chance that the mechanism will jam when larger tissue masses are intended to be disaggregated. The sequential treading action also reduces the chances of the bag 10 rupturing.

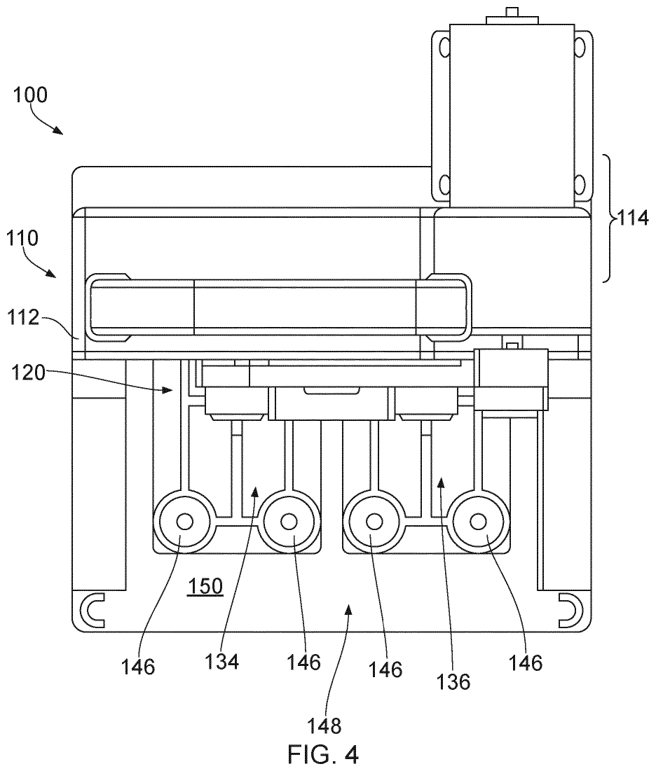

FIG. 4 is a plan view of the device 100 described above, but no bag 10 is in place in this view. In particular, the relative side-by-side positions of the foot assemblies 134 and 136 can be seen, which are spaced and have a collective area viewed in plan, which area is about equal the area of the bag 10 when laid flat, but a difference in areas of about plus or minus 10% of the area of the bag 10 has utility.

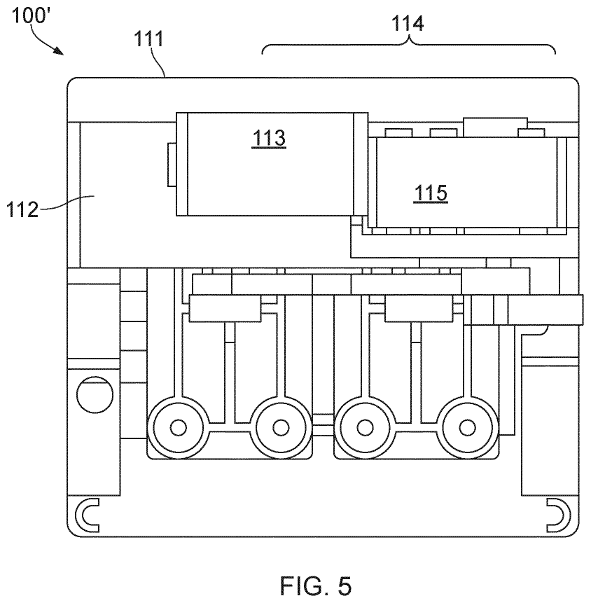

FIG. 5 shows another plan view of a device 100′ which is similar in construction to the device 100 described above, but in this alternative the motor 113 of the motor unit 114 is arranged transversely to the output shaft of its gearbox 115 by the use of a 90 degree gearbox 115, so that the motor 113 does not protrude beyond a backwall 111 of the device 100′. Thus, this device 100′ can fit into a smaller freezer volume if needed.

During the above-mentioned disaggregation processing, the forces exerted by the foot assemblies 134 and 136 are reacted by the heat transfer plate 150. This means that the sample bag 10 is pressed against the contact surface 151 of the plate 150 during processing, providing good surface contact between the sample bag 10 and the plate's surface 151, and consequently improved heat energy transfer.

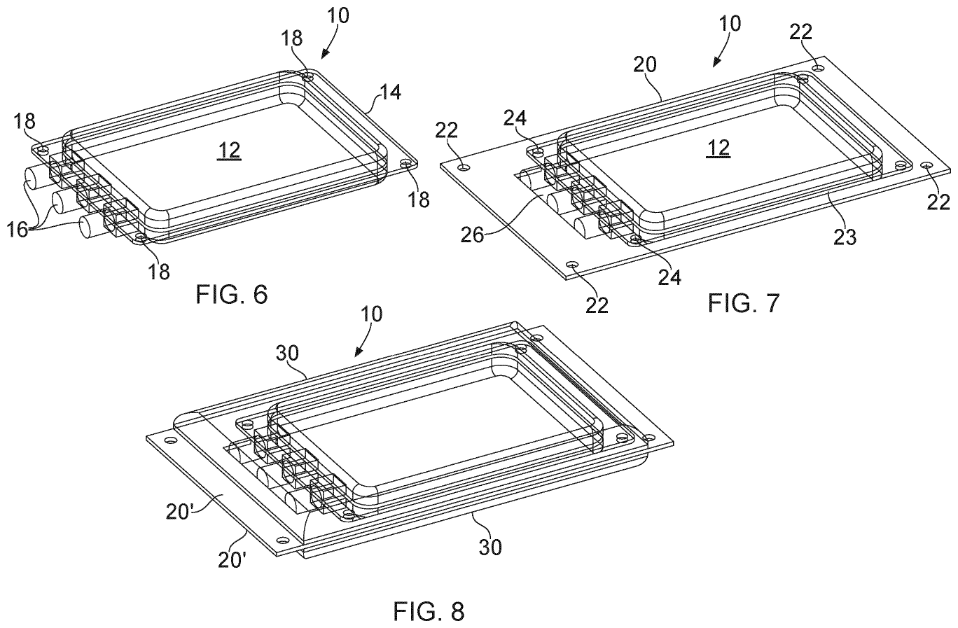

FIGS. 6, 7 and 8 show different embodiments of the flexible sample bag 10 mentioned above. The bag in use is slid into place in the receiving area 148 in the device 100 or 100′and sits under the two feet 134 and 136 mentioned. Thus, the bag has a generally flat construction, of about up to 12 mm thickness, with some additional compliance in order to fit tissue samples therein. As can seen from FIG. 6 one construction of a bag 10 is shown formed from two layers of plastic material sealed only at their periphery 14 to form a central cavity 12, and ports 16 for access into the cavity 12. The bag may be formed from EVA. In use it is preferred that the ports 16, or at least one of them, is/are large enough, i.e. about 10 mm in diameter or larger, to accept a sample which if necessary has been chopped into small pieces and passed into the bag cavity 12 by means of a syringe. However, it is also possible to include a so called ‘zip-lock’ access at the end of the bag opposite the ports, such that large tissue samples can be put into the bag and the bag is then re-sealed. The ‘zip-lock’ can be folded over one or more times to make a seam, held folded inside a resilient channel or by means of another clamp or clamps (not shown) to reduce the chance of leakage. The bag 10, can, as an alternative, be opened and tissue can be added. The bag can then be heat sealed with its contents in place. The bag 10 includes corner apertures 18 for locating the bag in the device in use and holding it in place during treading. Whilst the drawings show a bag 10 with one cavity 12, it would be possible to provide a bag having more than one cavity, for example, two, three, four or five cavities, for example each of the plural cavities being elongate and having an initially open, heat sealable end, and a sealable port at its other end for the introduction of reagents such as a disaggregation enzyme, and for withdrawing the disaggregated sample once the disaggregation is complete or substantially complete.

FIG. 7 shows the bag 10 of FIG. 6 mounted in a locating frame 20 by means of pegs 24 on the frame which fit into the corner apertures 18. The frame 20 is an alternative way of locating and holding the bag 10 in place within the device 100/100′. The frame 20 includes location holes 22 which cooperate with the device for locating and holding the bag in place during treading. The frame has an inner open window 26 with a smooth rounded inner edge 23, to accommodate the cavity 12 and treading feet 134 and 136 in use. The frame 20 makes loading and unloading of the bag 10 into and out of the device 100/100′ easier.

FIG. 8 shows an alternative frame 20′ which has two generally symmetrical halves each similar to construction of frame 20. Each frame half has additionally a flexible shell 30 moulded to the frame 20′, such that the two halves come together like a clam shell enveloping the bag 10. The top and bottom flexible shells act as a bund if the bag 10 inside ruptures in use. This feature is particularly useful for infectious tissue samples.

Yet another alternative, not shown, a simple bag-in-bag arrangement could be employed to contain leaks. In yet another alternative, the bag may include a base which has resilient (at least at room temperature) separate wells, such that aliquots of sample can be removed without using the whole sample, for example after freezing as described below. Alternatively, a sealable bag may be further heat sealed into portions for allowing the separation of the sample.

The processing of a sample put into the bag 10 can in one example largely follow the steps described in WO2018/130845. In this arrangement the sealed bag 10 containing tissue is suspended in an aqueous solution which may contain digestive enzymes such as collagenases and proteases to accelerate the breakdown of the tissue, introduced into the bag via a port 16. The bag is here placed on the plate 150 and warmed from, for example, an external heat source to approximately 35° C. to accelerate the rate of tissue digestion. One important difference proposed here is that a single sample processing bag is employed, and digestive enzymes can be introduced through one of the ports 16 in the bag prior to or during disaggregation. The heat transfer plate 150 can be used to introduce heat energy into the bag by heating the plate on its underside to provide the desired temperature in the bag for enzymatic action. That heat could conveniently come from an electrically heated warming plate, or electric heating elements in or on the plate 150. The amount of disaggregation action will depend on numerous parameters, for example the size, density and elasticity of the initial tissue sample, and so the time for disaggregation and the rate of treading will vary significantly. Too long or overly vigorous treading could lead to decreased cell viability. Thus, the motor unit speed and the disaggregation period is important. One option to address this problem is to time the processing according to a look-up table which includes times and output speeds required to disaggregate similar samples. Another option is to measure the instantaneous electrical power or electrical energy over time needed to perform the disaggregation processing, or to measure the force or stress exerted on the plate 150 or another part of the mechanism, and to stop after a predetermined threshold has been reached, to indicate that the sample has been sufficiently disaggregated. As the power/forces/stresses reduce the disaggregation is closer to completion. Another option is to measure light absorbance through the bag—the, greater the absorbance, the closer the sample is to complete disaggregation. Once disaggregation is complete the bag contents can be transferred, and the cells or other constituents of interest can be separated and put back into a fresh bag for freezing in the device 100/100′. Alternatively, and preferably the whole disaggregated materials can be left in the bag and device for freezing. A cryoprotectant is introduced in to the bag through a port 16.

Another difference between the present methodology and that described in WO2018/130845 is that once a cryoprotectant is introduced, the device with the disaggregated sample and cryoprotectant in the bag is mounted (or remains in) the device, and the whole device is mounted in the freezer 40 as described above. The base of the freezer is cold and so draws heat energy from the bag 10 via the heat transfer plate 150. To control the formation of ice and prevent supercooling of the sample while the bag it is being cooled, it can be massaged by the feet 134 and 136, in the manner described above, albeit at a slower rate than for disaggregation, to control ice nucleation and so increase the viability of the cells after thawing. Electrical energy can be supplied to the motor unit 114 via a wire conductor to maintain motion of the mechanism 120 inside the freezer, e.g. freezer 40 (FIG. 1).

Since the device is removeable from the freezer, cleaning after use is made easier.

When required for use, the frozen disaggregated samples in a bag 10 can be thawed rapidly in the device 100/100′ by further external heating of the plate 150, and/or by partially immersing the device 100/100′ in a warmed water bath, maintained at about 37° C., and the cryoprotectant removed. In each case the bag can be massaged during thawing. If the enzymes are still present, they too can be removed if needed, for example by means of filtering. Generally, they will have had little or no effect on the cells during cryopreservation because their action is halted at low temperatures. All the process manipulations, warming, disaggregation, cooling, freezing and then thawing occur with the sample in the same sealed flexible bag 10, and may be performed in a single device. This is not only time and space efficient, but it enables a single record to capture everything that happened to the sample during processing, e.g. temperatures, durations, disaggregation speed, freezing protocol, and lessens the chance for errors, such as a sample spending too much time in an uncontrolled environment between processing machines.

More specific examples of the apparatus and techniques used in tissue sample processing and freezing are given below.

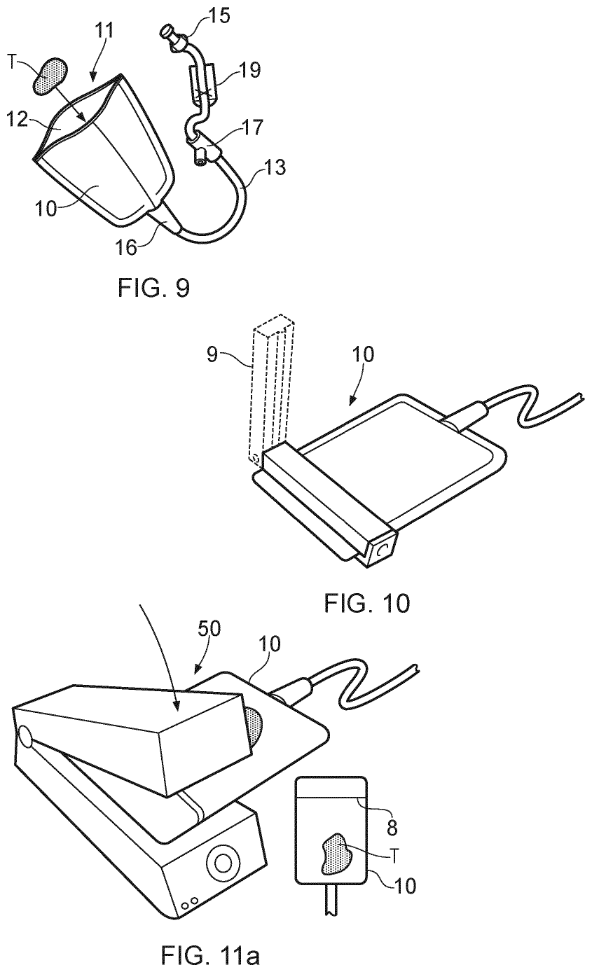

FIG. 9 shows an example of a bag 10 formed from a thermoplastic material such as EVA or PVC film and having an opening 11 for accepting the tissue sample T. The bag includes tubing 13 attached to the one or more ports 16 (FIG. 6) which tubing includes one or more branches 17, compression valves 19, and standard Luer-type connectors 15. The single tubing line shown is merely illustrative—the bag 10 may include additional parallel tubing connected via plural ports 16.

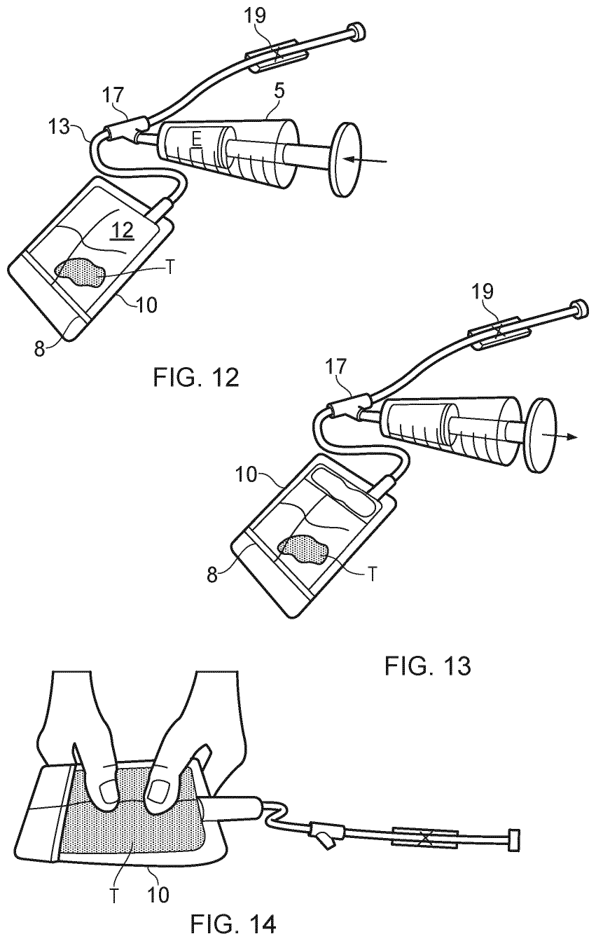

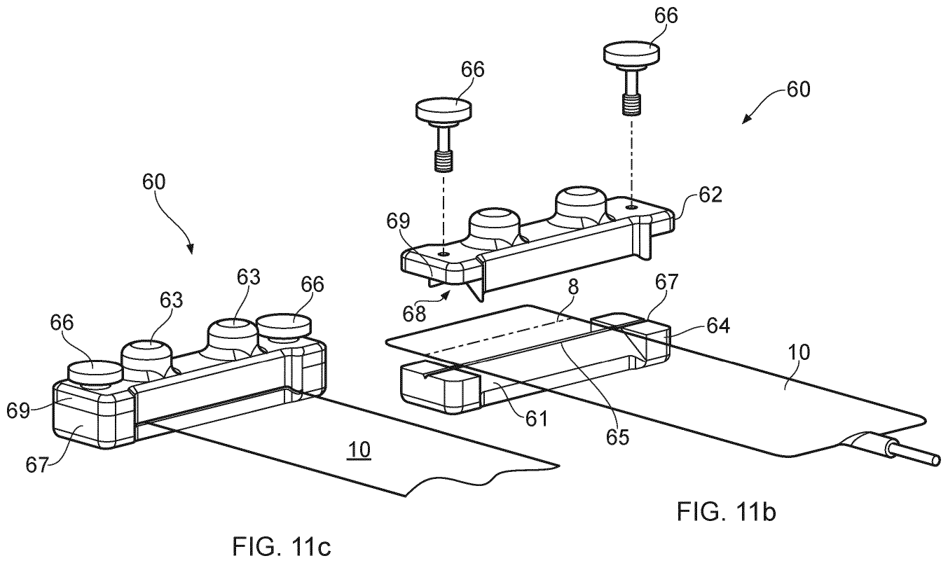

Once the tissue T is inside the bag 10, the opening 11 can be sealed by a mechanical clamping seal 9, shown closed and sealed in FIG. 10, and shown open in chain dotted lines in the same Figure, and/or by means of heat sealing using a heat sealing machine 50 as shown in FIG. 11a, to produce a heat-sealed closure strip or strips (for example plural parallel strips) 8, each method forming the sealed cavity 12 (FIGS. 6, 7 and 9).

An alternative or additional means for sealing a bag 10 is shown in FIGS. 11b and 11c. As shown in FIG. 11c, the bag 10 after heat sealing at seal 8 can be clamped in a two piece clamp 60, which comprises a top bar 62 and a bottom bar 64 forced together by a pair of screws 66. FIG. 11b shows the clamp 60 in an exploded condition, but in use the screws 66 need not be completely removed from the remaining clamp prior to insertion of the bag 10. The top bar 62 has a tapering recess 68, in which sits a complementary wedge shaped formation 61 when clamped. The recess and wedge concentrate the clamping forces at the apex of the wedge 65, providing higher clamping forces at the apex than could be achieved by flat clamping faces. For even more clamping force, the apex 65 has a small channel 67 at its peak, which is met in use by a complementary ridged formation 69, in the top bar. The forces are sufficient to negate the need for the heat seal 8, although such a seal has been illustrated for extra security. The clamping force is further enhanced by the thickness and stiffness of the top and bottom bar which do not readily bend, and so maintain the clamping force exerted by the screws 66. FIG. 11c shows the clamp 60 in a clamped condition. Protrusions 63 meet with features of the treading device 100/100′ or 200 (as described below) to inhibit movement of the clamp, and consequently the clamped bag 10 during treading. The outer periphery and height of the clamp 60 is of a sized and shape to fit in a complementary part of the sample receiving area 148 (or 248 FIG. 22 et seq), and so afford further location of the clamped bag 10 during treading. Although not illustrated, the clamp 60 may incorporate also an additional frame 20, 20′ as shown in FIGS. 7 and 8, and such that the clamp is rigidly mounted to one end of the frame and the port(s) 16 (FIGS. 6 and 9) are supported at the other end of the frame.

With reference to FIG. 12, in use, once sealed, a digestive enzyme E can be introduced into the cavity 12 via the tubing 13, for example by injecting the enzyme into the bag using a syringe 5 attached to the branch connection 17. By holding the bag in an upright orientation, air can then be removed from the cavity 12 by withdrawing the piston of the syringe 5 as shown in FIG. 13. Initial mixing of the enzyme E and tissue T can be made by hand as shown in FIG. 14.

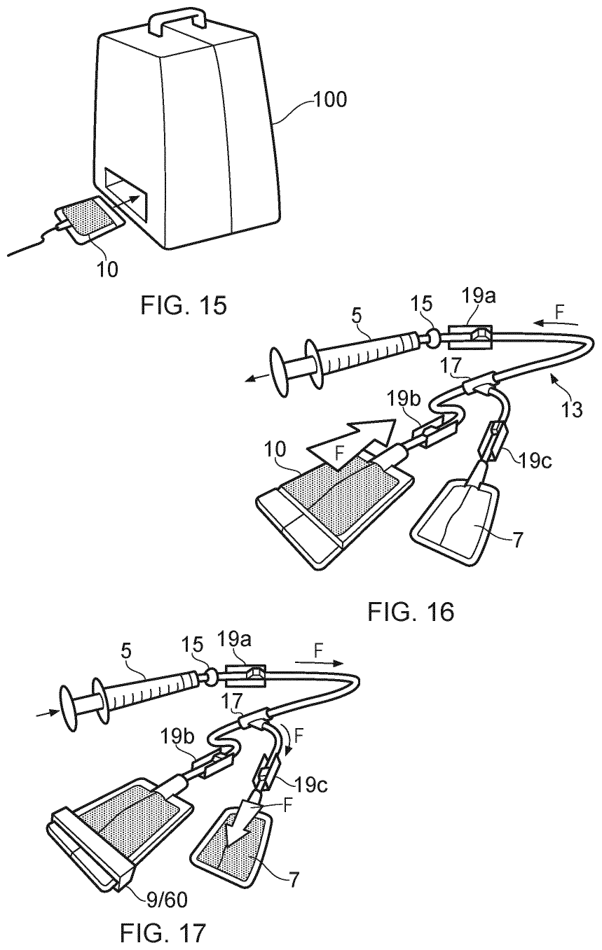

Loading of the bag 10 into the treading device 100 for disaggregation can then be commenced, either with or without the frame 20/20′ and bunding cover 30, as illustrated in FIG. 15.

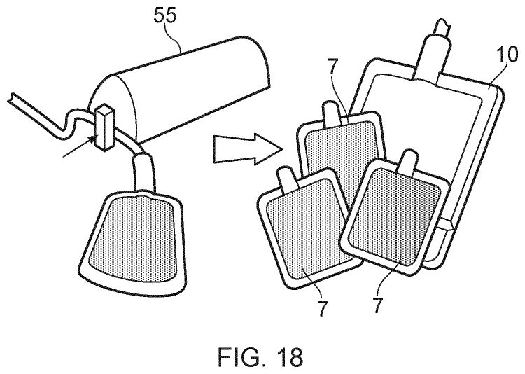

The disaggregation process then takes place as described above. Once complete, which may take between several minutes and several hours for example around 10 minutes to 7 hours, preferably 40 minutes to 1 hour, the disaggregated liquefied sample may be subdivided in to aliquots, for example using the bag set described above, and an additional sample aliquot bag 7, as shown in FIG. 16, connected to the branch 17. In that instance a syringe 5 is used to draw the liquefied sample out of the bag 10 in the direction of arrows F, valves 19a and 19b are open and valve 19c adjacent the sample aliquot bag 7 is, closed. Once sufficient sample has been withdrawn into the syringe 5, valve 19b is closed, valve 19a remains open, and valve 19c is opened. The syringe is then used to force the liquids in the direction of arrow F in FIG. 17, into the sample aliquot bag 7. The tubing 13 of aliquot bag 7 can be heat sealed by means of a clamp heat seal machine 55 and shown in FIG. 18. That process can be repeated until sufficient aliquots are obtained or until the is no more sample left Bag may be partially divided already to make sealing off each compartment simpler.

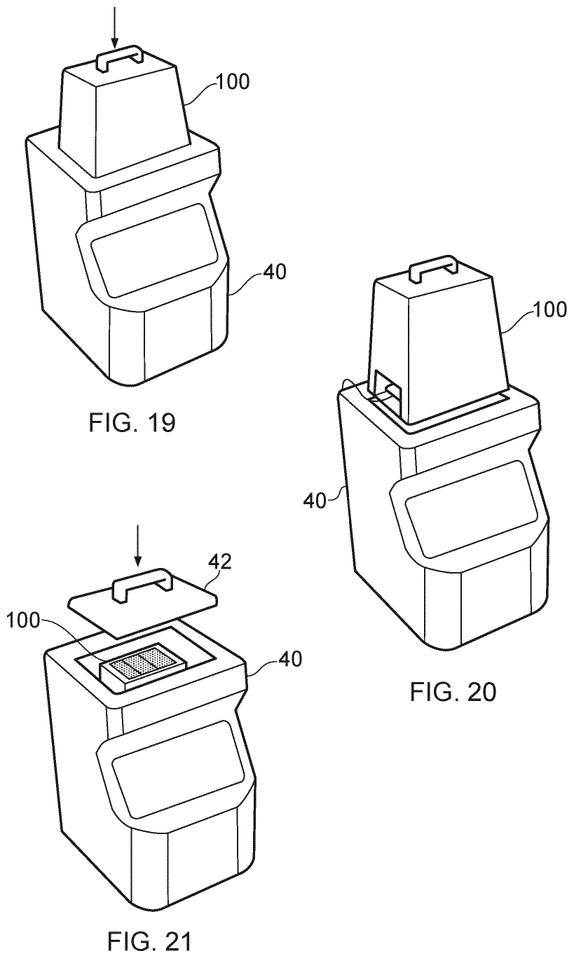

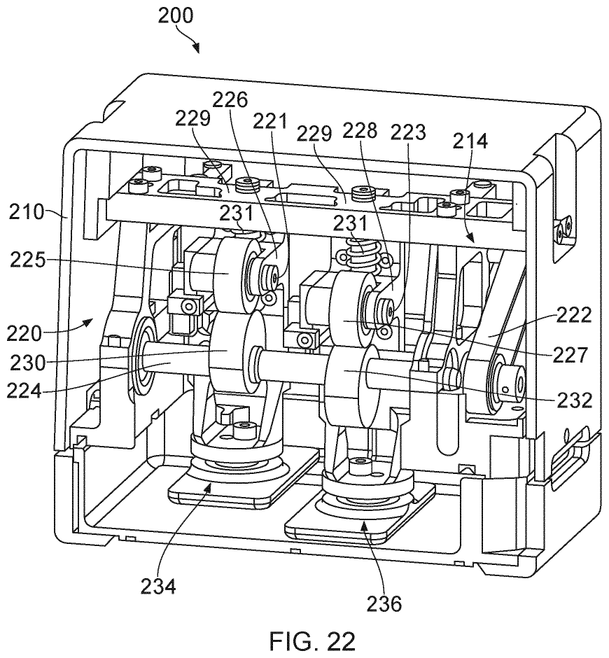

As described above, the sample bag 10, can remain in the treading device 100 (FIG. 15) and the treading device can then be loaded into a controlled rate temperature change device, in this case the freezer 40 as shown in FIG. 19. That technique allows treading to continue during freezing, to inhibit ice crystals forming, although in practice the bag 10 can be removed before freezing, and the freezer 40 then acts only to cool the sample through the heat transfer plate during treading. In the alterative, the aliquot sample bags 7 can take the place of the whole sample bag 10. In another alternative, the freezer 40 can be used to gently cool the unprocessed or processed sample to around 4 degrees Celsius by mounting the treading device 100 on top of the freezer 40 with its lid open so the base 150 is cooled, as shown in FIG. 20. In another alternative it is possible to remove the base 150 and put that into the freezer, with the freezer lid in place, as shown in FIG. 21. In yet another alternative, not shown, the bags 10, or 7 can be frozen directly in the freezer 40.

The invention is not to be seen as limited by the embodiments described above, but can be varied within the scope of the appended claims as is readily apparent to the person skilled in the art. For instance, the treading mechanism described above is preferred because it provides wholly pivoting mechanical interconnections which are less likely to jam in cold conditions than sliding surfaces, but that mechanism could be replaced with any mechanically equivalent means for treading two or more feet sequentially. The flat feet described may be replaced with roller feet, where the treading motion is from side to side rather than up and down. The treading described, or its mechanical equivalent, is preferably at a rate of 2 or 3 treads for each foot per second to optimise disaggregation and maximise cell recovery, and is a steady treading, but the treading could be quicker or slower, or intermittent, for different cell types.

Since the device 100/100′ is intended to be placed in a freezer and subjected to extremely low temperatures (e.g. minus 80 degrees Celsius or lower), the use of metal parts, particularly those parts like springs 146 is preferred since polymeric parts become much more rigid at low temperatures. Also, tightly fitting parts, like pistons and cylinders, can become jammed or ill-fitting at very low temperatures so simple pivotable linkages like the mechanism 120 described are preferred.

FIGS. 22, 23 and 24 show an alternative treading device 200, which is similar in size and function to the device 100 described above. The device 200 has certain differences which are described in more detail below.

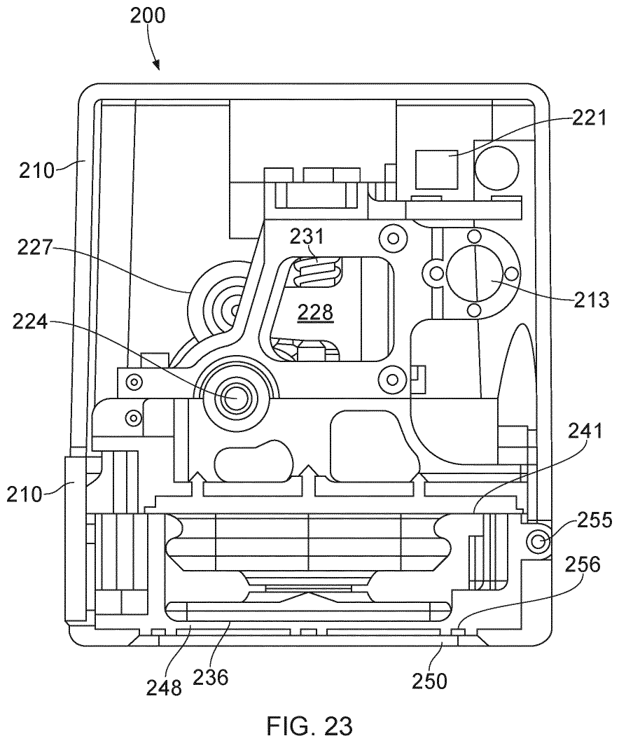

Referring to FIG. 22, the principal difference between the device 100 and the device 200 is that the device 200 has a treading mechanism 220 which is different to the mechanism 120 of device 100. Two treading feet 234, 236 driven in a cyclic alternate treading motion, similar to the motion shown in FIGS. 2 and 3, by a 24 volt DC electric motor 213 (FIG. 23) which is part of an electric motor unit 214 which has a rotary encoder providing feedback to a controller 221 (FIG. 23) for monitoring and controlling the speed of the treading motion. The motor drives a cam shaft 224 via a toothed belt 222. The cam shaft includes a pair of cams 230, 232 offset at 180 degrees, in this instance, each profiled with a cycloidal shape to provide simple harmonic motion of the cam follower. Each cam is operable to move a cam follower assembly including an associated elastomeric follower wheel 225, 227 which rides over the cam's profile, a follower wheel axle 221, 223 in force transmitting relationship with a sprung follower carriage 226, 228. Each carriage 226,228 slides in a linear guide 229, and a respective foot 234, 236 is connected to the carriage; Each assembly is forced upwards in turn by a respective one of the follower wheels as it rides the cam profile away from a treading condition together with the foot, as the respective cam is rotated by the motor against the urging force of a return spring 231. As the cam is rotated further, and the cam profile recedes, the spring 231 associated with each follower assembly forces the assembly and foot downwards with a treading force.

Thereby, the treading force is limited to the spring rate of the associated follower assembly spring 231 and not the power of the drive motor 1. The force applied to the bag is, in use, limited by the springs because the mechanism drives the feet up and the springs push them back down. This makes sure that:
a. the motor cannot stall (regardless of tumour size or texture);
b. the sample is not compressed with excessive force and the bag will not split;
c. the maximum pressure applied to the bag is lower than the pressure tested during bag manufacture; and
d. As described below, a hinged bag receiving area 248 can accept a sample bag and any clamp used, without necessarily pre-positioning the feet. In other words, the feet can be in any position when accepting a bag, because the hinged sample area 248 is closed against the feet, and if needed any sample can at that time be compressed by the feet as the hinged area is closed against the feet.

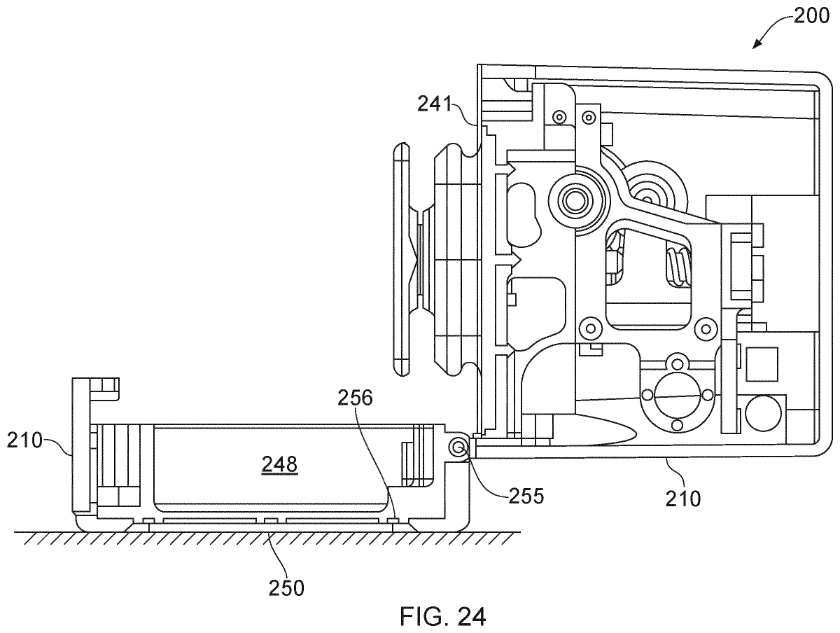

Referring also to FIGS. 23 and 24, the device 200 further includes a flexible sealing membrane 241 extending from a device housing 210 to the upper parts of the two feet 234, 236 which provides a fluid resistant and dust seal between the soles of the feet and the remaining parts of the treading mechanism 220. That arrangement inhibits mechanism contamination, should the compressed bag split in service. Whilst a membrane 241 is preferred, the feet could slide in seals, such as lipped seals mounted to a partition dividing the mechanism 220 from the bag area 248, and achieving similar inhibition of contamination of the mechanism should that be needed.

The device 200 further includes heat transfer plate 250, which performs the same function as the heat transfer plate 150. This plate 250, however, is hinged to one side of the housing at hinge 255 (FIG. 24), so that insertion and removal of the bag to be trodden (as shown in FIGS. 6, 7 and 8) is easier. The heat transfer plate 250 includes a temperature sensor 256 which allows the temperature of the plate 250 and the bag receiving area 248 to be monitored and recorded by the controller, for quality control. The plate 250 has first and second surfaces 251 and 252 with the same function as the surfaces 151 and 152 described above.

Each foot is adjustable in height relative to a heat transfer plate 250 of the device 200 and an indication of its movement is monitored also by the controller. Thus, even though the rotary encoder may indicate that the motor is turning, a mechanical failure, such as a failure of the toothed belt 222, may still be detected by the controller, and a suitable action can be implemented, such as raising an alarm.

The device 200 has the same external dimensions as the device 100, and the device's housing 210 is intended to slide inside the controlled rate freezer 40 with the freezer lid in place as described above and illustrated in FIG. 21.

For convenience, terms such as upper, lower, up and down, and more descriptive terms such as feet, tread and treading have been used to described the invention shown in the drawings, but in practice, the device shown could be oriented in any manner such that those terms become for example inverted or less descriptive in that new orientation. Therefore, no limitation as to orientation should be construed by such terms or equivalent terms.

The invention provides A device (100/100′) for the disaggregation of tissue samples into individual cells or cell clumps in a closed flexible bag (10), the device including a mechanical disaggregation mechanism (120) and a tissue sample bag receiving area (148), said device further including a heat transfer plate (150) for transferring heat energy to or from the area (148), the plate having a first plate surface (151) adjacent the area (148) and an opposing surface (152) exposed to external thermal influence which faces away from the area (148).

1. A device for the disaggregation of tissue samples into individual cells or cell clumps in a closed flexible bag, the device including a mechanical disaggregation mechanism and a tissue sample bag receiving area, said device further including a heat transfer plate for transferring heat energy to or from the area, the plate having a first plate surface adjacent the area and an opposing surface exposed to external thermal influence which faces away from the area the disaggregation mechanism including plural treading feet each urged toward the first plate surface with generally linear motion only by force from a respective resilient member, and each foot being further movable away from the first plate under influence of a mechanical member which is arranged also to compress said respective resilient member during said movement away from the first plate.

2. The device as claimed in claim 1, wherein the mechanism includes two or more feet arranged to tread sequentially the tissue sample bag receiving area.

3. The device as claimed in claim 2, wherein said linear motion is motion toward and away from the bag receiving area in a direction generally perpendicular to the first plate surface.

4. The device as claimed in claim 2, wherein said mechanism includes two cams each having lobes arranged at a 180 degree rotational separation.

5. The device as claimed in claim 1, wherein the feet have a collective treading area about equal (up to plus or minus 30%) to the area of the bag intended to be trodden, when such a bag is laid flat.

6. The device as claimed in claim 2, wherein said feet when moving toward the area, act to push a sample bag directly onto the adjacent first surface of the heat transfer plate.

7. The device as claimed in claim 1, wherein said heat transfer plate has a heat conductance of 100 W/m K or more and preferably above 200 W/m K measured at 20 degrees Celsius.

8. The device as claimed in claim 1, wherein the finally urged position of the feet above the first surface is adjustable.

9. The device as claimed in claim 1, wherein the mechanism is within or substantially within a housing and the tissue sample bag receiving area is separable or moveable relative to said housing for example by means of a hinge.

10. The device as claimed in claim 1, wherein the mechanism is sealed from said feet for example by means of a flexible membrane, or sliding seal.

11. A system for cryopreservation of disaggregated cells, the system comprising the device for the disaggregation of tissue samples into individual cells or cell clumps removably disposed in a controlled temperature rate change device as such a warmer/freezer, the device having mounted or mountable therein one or more closed flexible bags for containing samples for disaggregation or disaggregated by said device, the device including a mechanical disaggregation mechanism and a tissue sample bag receiving area, said device further including a heat transfer plate for transferring heat energy to or from the area, the plate having a first plate surface adjacent the area and an opposing surface which faces away from the area exposed to a thermal influence of the freezer, the disaggregation mechanism including plural treading feet each urgable toward the first plate surface, for example one after the other.

12. A method for disaggregating tissue samples into cells or clumps of cells, the method comprising the following steps in any suitable order:
a) providing a tissue sample sealed or substantially sealed in a flexible sample bag;
b) providing a device including a mechanical disaggregator, including a sample bag receiving area, and including a heat transfer plate having a first surface adjacent the area and an opposing surface exposed to external thermal influence which faces away from the area, and optionally including any one or more of the remaining features of the device of claim 1;
c) subjecting said tissue sample to disaggregation in the device, and
d) transferring heat energy into or out of the bag via said plate, by means of disposing the device in a controlled temperature rate change device.

13. The method as claimed in claim 12, wherein step d) includes initially introducing heat energy into the bag contents via the plate to aid enzymatic disaggregation or to thaw the contents of the bag.

14. The method of claim 11, wherein step d) includes removing heat energy for cooling the bag contents, or for freezing the contents of the bag, and optionally including the introduction of a cryoprotectant prior to said freezing.

15. The method as claimed in claim 12, wherein said disaggregation device exerts a cyclic pressure on the bag, for example from zero to up to about 6N/cm2 or any range between zero and 6N/cm2.

16. (canceled)

17. A tissue sample receiving bag comprising one or more flexible plastics cavities formed with a generally rectilinear periphery with the cavity or cavities within the periphery, and at one side of the periphery is formed one or more sealable or closable access ports, optionally the periphery also including apertures for location and securing of the bag during treading of the bag

18. A tissue sample receiving bag comprising two plastics layers sealed together around the majority of their edges to form said periphery, and having a region of the periphery unsealed, to form an opening in the bag for the receipt of a sample into the bag, and having an additional closable opening in the form of a tube port.

19. The tissue sample receiving bag as claimed in claim 18, further including a clamp for sealing the opening in use, said clamp having complementary clamping members suitable for location within a sample bag receiving area.

20. The tissue sample receiving bag as claimed in claim 17, further including a frame having an opening of a size that accepts the cavity or cavities and wherein at least a portion of the periphery overlaps the frame, the frame and periphery having complementary formations for holding said at least a portion the periphery to the frame.

21. The tissue sample receiving bag as claimed in claim 20, wherein the frame includes upper and lower portions that come together in use, each portion further including a flexible cover encapsulating the cavity for acting as a bund around the cavity.
